(19) United States
(12) Patent Application Publication
(10) Pub. No.: US 2025/0344738 A1
(43) Pub. Date: Nov. 13, 2025

(54) NUTRITIONAL COMPOSITION WITH IMPROVED SEGREGATION RESISTANCE

(71) Applicant: N.V. NUTRICIA, Zoetermeer (NL)

(72) Inventors: Martijn Johannes VAN DER HOEVEN, Utrecht (NL); Anika Kirsten KROHN, Utrecht (NL); Francisco Hernan BLANCO HEDDERICH, Utrecht (NL)

(73) Assignee: N.V. NUTRICIA, Zoetermeer (NL)

(21) Appl. No.: 19/274,705

(22) Filed: Jul. 21, 2025

Related U.S. Application Data
(63) Continuation of application No. PCT/EP2024/053076, filed on Feb. 7, 2024.

(30) Foreign Application Priority Data
Feb. 7, 2023 (EP) 23155330.6

(51) Int. Cl. A23L 33/00; A23L 33/125

(52) U.S. Cl. CPC A23L 33/40; A23L 33/125

(57) ABSTRACT

The present invention relates to a powdered nutritional composition comprising (i) milk formula particles comprising lipid, protein, digestible carbohydrates, and (ii) human milk oligosaccharide (HMO) particles, wherein the nutritional composition is selected from an infant, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm
   and wherein the HMO particles comprise at least 2′fucosyllactose (2′FL). The invention also relates to a process to prepare the composition and uses thereof.

### FIELD OF THE INVENTION

The present invention relates to powdered nutritional compositions that have an improved segregation resistance and comprise milk formula particles comprising lipids, protein, digestible carbohydrates, and human milk oligosaccharides (HMOs) particles. The nutritional composition is in particular an infant formula, a follow-on formula or a growing up milk that comprises lipid globules having a volume weighted mode diameter of at least 1.0 μm, preferably wherein at least 40 vol % has a diameter between 2 μm and 12 μm.

### BACKGROUND OF THE INVENTION

Infant or follow-on formulae are commonly used when breastfeeding is inadequate or unsuccessful for medical reasons, or because of a choice not to breastfeed. Commercial infant formulae are commonly used to provide supplemental or sole source of nutrition in early life. These formulae comprise a range of nutrients to meet the nutritional needs of the growing infant, and typically include fat, carbohydrate, protein, vitamins, minerals, and other nutrients helpful for optimal infant growth and development.

Nutritional compositions for infants and young children are often sold as powders to be reconstituted with water or in some instances as ready to drink or concentrated liquid compositions. Those compositions are intended to cover most or all the nutritional needs of the infants or young children.

Human milk lipids have a distinct physical structure composed of large lipid globules with an average mode diameter of about 4 μm existing of a triglyceride core coated by a tri-layer of membranes, the milk fat globule membrane (MFGM). The diameter of lipid droplets in standard infant formula is about 0.3 to 0.5 μm due to the industrial processing procedures to achieve stable and reproducible end products and is not surrounded by MFGM but mostly by proteins such as casein. Standard commercially available formulae also mostly contain vegetable oils and have small lipid droplets with proteins adhering to the surface. WO 2021008982 discloses such composition comprising lipid, protein and digestible carbohydrates for inducing satiety; wherein the lipid comprises: i) 30 to 90 wt. % vegetable fat, and ii) 10 to 70 wt. % mammalian milk fat, wherein all wt. % are based on total lipid of the composition, characterized in that the lipid is present in the form of lipid globules with the volume % of lipid globules with a diameter below 2 μm is above 60%, preferably above 70%, more preferably above 80%, most preferably above 90%. The volume percentage of lipid globules with a diameter below 1 μm in the composition was 80% and the volume % of lipid globules with a diameter below 2 μm was 92%. The mode diameter of the test product is 0.46 μm, based on volume. It is disclosed in WO′982 that the composition may further comprise one or more human milk oligosaccharides (HMOs), most preferably 2′FL. Furthermore, the lipid globules may comprise a coating comprising phospholipids (at least 0.5 wt %), such as phospholipids originating from MFGM.

Human milk is known to contain a large amount of indigestible human milk oligosaccharides representing the third largest solid component of breast milk and acting as prebiotics. Various infant formulae supplemented with prebiotics, such as mixtures of fructooligosaccharides (FOS) and galactooligosaccharides (GOS) for example are commercially available. However, there is an ongoing focus on the addition of human milk oligosaccharides (HMOs) to infant formulae to provide for nutritional composition that resemble human breast milk more and provide for the associated health effects.

It has now been found that in nutritional compositions with fat droplets with a low volume weighted mode diameter such as in the above WO′982, like in most commercially available infant milk formulae wherein the majority of the fat droplets have a diameter of below 1 μm, undesired segregation of HMOs occurs. Powder segregation is an undesired phenomenon, as it is crucial for infant formula that each scope of powder provides the same amount of nutrients. There should be no difference between the powder in the top and the powder in the bottom of a pack.

Powder segregation may be caused by the differences in size, shape or density of the powder particles. Particle size is probably the most significant contributor to segregation.

Small particles may move downwards through the mass falling into the spaces between larger particles in powder formula. At the same time, the larger particles move upwards in the so called ‘Brazil Nut effect’ as voids are created and then filled in with the smaller particles forcing the larger particles to move upwards. This type of segregation, called percolation, typically takes place after packaging of the powder, e.g. during transport of the packaged product on its journey from the factory to the consumer's home.

Infant formula with lipid globules with an architecture more similar to the lipid globules in human milk have been described. In WO 2011115476, WO 201027258 and WO 2012173467 the use of specifically designed lipid component with optimal fatty acid profile, an enhanced portion of the palmitic acid residues at the sn-2 position and present as lipid globules with a certain size and/or coating is disclosed for an early in life diet for improving the development of a healthy body composition, in particular prevention of obesity later in life, and a body weight development more similar to breast fed infants.

These disclosures do not describe the effect of these formulations on segregation behavior especially when compared to formulations without these large fat droplets when HMOs are blended into them. While WO 2015067325 discloses that adding micronized lactose or another micronized carbohydrate to a nutritional composition with large fat droplets improves the flow of such product, WO′325 is silent on segregation properties. Regardless, HMOs and micronized lactose have clearly distinct properties both in terms of physical parameters as from a functional perspective, HMOs do not have the same micronized particle size and are contrary to lactose indigestible for humans.

The present invention provides infant formulae with lipid globules with an architecture more similar to the lipid globules in human milk further comprising HMOs and wherein undesired segregation is prevented.

### SUMMARY OF THE INVENTION

A study on the segregation behavior of powdered milk formula compositions with a human milk oligosaccharide (HMO) was conducted. The inventors of the present invention observed that while regular compositions with fat droplets with a low volume weighted mode diameter below 1 μm suffer from undesired segregation, it was unexpectedly found that this segregation is reduced when using a composition in which the fat droplet diameters are increased, namely products in which the lipid globules have a volume weighted mode diameter of more than 1 μm and/or wherein a substantial part of the globules have a diameter of at least 2 μm and up to 12 μm.

The inventors have found that milk formula particles comprising said large lipid globules reduce the powder segregation in a powdered nutritional composition comprising both said milk formula particles and HMO particles, when the milk formula particles and HMOs differ in particle size.

The inventors now found that the milk powder particles of nutritional compositions according to the invention with lipid globules having a volume weighted diameter and have a powder particle size distribution that are both larger than the powder particle size distribution and lipid globules mode diameter of regular nutritional compositions combined with HMO particles. The HMO particles have a different, smaller, particle size distribution than the milk formula particles according to the invention comprising the large lipid globules. A larger particle size distribution of the milk formula particles was surprisingly found not leading to an increase in segregation in compositions also comprising HMO particles. This is very remarkable as the difference is particle size in the nutritional compositions of the invention is larger than in the state of the art nutritional compositions when containing these HMOs particles and hence goes against the expectations derivable from the physical theories on powder segregation.

Although the inventors do not wish to be bound by theory, it is believed that the large lipid globules may have some form of interaction with the HMOs and thereby prevent powder segregation.

Accordingly, the present invention provides for a powdered nutritional composition comprising (i) milk formula particles comprising lipid, protein, digestible carbohydrates, and (ii) HMO particles, wherein the nutritional composition is selected from an infant, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm, and wherein the HMO particles comprise at least 2′FL.

The present invention provides for a powdered nutritional composition comprising milk formula particles comprising lipid, protein, digestible carbohydrates, and HMO particles,
wherein the nutritional composition is selected from an infant formula, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm, wherein the HMOs comprise at least 2′FL, and wherein
the milk formula particles have a powder particle size distribution comprising a Dx(10) of at least 60 μm and/or a Dx(50) of at least 200 μm and/or a Dx(90) of at least 400 μm.

The present invention in addition provides a process to prepare such nutritional compositions, the products obtainable therewith including after reconstitution, and use thereof.

The invention also pertains to the use of milk formula particles to reduce powder segregation in a powdered nutritional composition, said nutritional composition comprising (i) milk formula particles comprising lipid, protein, digestible carbohydrates, and (ii) HMO particles, wherein the nutritional composition is selected from an infant, follow-on formula and growing up milk, wherein said nutritional composition is not human milk, wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm
and wherein the HMO particles comprise at least 2′fucosyllactose (2′FL).

In a preferred aspect of the invention the use of the milk formula particles further pertains to (i) the milk formula particles of the nutritional composition having a powder particle size distribution comprising a Dx(10) of at least 60 μm and/or a Dx(50) of at least 200 μm and/or a Dx(90) of at least 400 μm.

The present invention also provides nutritional compositions, preferably powdered nutritional compositions, comprising (i) milk formula particles comprising lipid, protein, digestible carbohydrates, and (ii) HMO particles,
wherein the nutritional composition is selected from an infant formula, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm,
wherein the HMO particles comprise at least 2′FL, for use in the promotion of metabolic health, the promotion of the development of good body composition, prevention of the development of obesity later in life, the promotion of balanced growth, the promotion of lean growth. the promotion of cognitive development, improving brain health, improving gut health, providing beneficial prebiotic effect, increasing immune cell function and immune health, preventing infections, or improving the recovery from infections, stimulating intestinal barrier functions/epithelial cell modulators and so to improve gut health and reducing the risk of gut health problems and/or improving a recovery of a gut health problem.

Reducing and preventing segregation of ingredients in a nutritional product has high benefits to customers. Segregation may be visual (larger particles of a composition being separated from smaller particles) and reduces customer liking of the product. When segregation is absent, consumers will know that for every bottle of product they make the composition will be substantially identical and so provide reliable and adequate nutritional quality. Consumers are more likely to discard a product that is suffering from segregation in the assumption that the composition is spoiled. The nutritional compositions according to the invention not suffering from this drawback are therefore better supported and used and do not suffer from the drawbacks associated with segregated nutritional compositions. Also, the product will be more stable and hence have an improved shelf-life property.

For some jurisdictions, the invention may also be worded as a method for (therapeutically) promoting metabolic health, promoting of the development of good body composition, preventing the development of obesity later in life, promoting balanced growth, promoting lean growth, promoting cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

For some jurisdictions, the invention may also be worded as the use of lipid, protein, digestible carbohydrates, and HMOs, wherein the HMOs comprises at least 2′FL in the manufacture of a nutritional composition which is an infant or follow-on formula or a growing up milk and comprises lipid globules and
a. the lipid globules have a volume-weighted mode diameter of at least 1.0 μm, and/or
b. at least 40 vol. % of the lipid globules having a diameter of 2 to 12 μm,
for the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

Worded differently, the invention also pertains to the use of the nutritional composition according to the invention for the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

### LIST OF EMBODIMENTS

1. Powdered nutritional composition comprising (i) milk formula particles comprising lipid, protein, digestible carbohydrates, and (ii) human milk oligosaccharides (HMOs) particles, wherein the nutritional composition is selected from an infant, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm and wherein the HMOs particles comprise at least 2′fucosyllactose (2′FL).
2. Nutritional composition according to embodiment 1 wherein the milk formula particles have a powder particle size distribution comprising a Dx(10) of at least 60 μm and/or a Dx(50) of at least 200 μm and/or a Dx(90) of at least 400 μm.
3. Nutritional composition according to embodiments 1 and 2 wherein HMO particles have a powder particle size distribution comprising a Dx(10) of at least 5 μm and/or a Dx(50) of at least 40 μm and/or a Dx(90) of at most 315 μm.
4. Nutritional composition according to the preceding embodiments wherein the (i) milk formula particles and the (ii) HMO particles have an Euclidean difference of more than 0.2.
5. Nutritional composition according to the preceding embodiments wherein the difference between the Dx(20) of the milk formula particles and the Dx(80) of the HMO particles is at least 10 μm
6. Nutritional composition according to any one of embodiments 1 to 5 wherein the lipid globules contain a coating comprising phospholipids and/or wherein the amount of phospholipids is 0.5-20 wt % on total lipid content.
7. Nutritional composition according to embodiment 6 wherein the phospholipids are derived from milk fat globule membrane (MFGM) or are provided as MFGM, preferably cow's milk MFGM.
8. Nutritional composition according to embodiments 1 to 7 wherein i) the lipid comprises linoleic acid and alpha-linolenic acid in a weight ratio of 2 to 20, and/or ii) the lipid comprises at least 10 weight percent palmitic acid based on total lipid, and at least 15 weight percent of this palmitic acid is esterified to the sn-2 position of a triglyceride based on total palmitic acid.
9. Nutritional composition according to embodiments 1 to 8, wherein the (ii) HMO particles are present in an amount of 0.1 wt %-2 wt %, preferably 0.3 wt % to 1 wt % based on dry matter weight.
10. Nutritional composition according to embodiments 1 to 9, wherein the lipid contains both vegetable fat and milk fat, preferably in a weight ratio of between 30:70 and 90:10.
11. Nutritional composition according to embodiments 1 to 10, wherein the amount of lipid is 10 wt %-30 wt % on dry matter, the amount of protein is 9.6 wt %-12 wt % on dry matter, the amount of carbohydrates is 40 wt %-70 wt % on dry matter and the amount of phospholipids is 0.5 wt %-3 wt % based on total fat weight.
12. Nutritional composition according to the preceding embodiments 1 to 11 wherein the HMOs comprise 2′FL and at least one selected from lacto-N-neotetraose (LNnT), para-lacto-N-neohexaose (para-LNnH), sialic acid, 3′ sialyllactose (3′SL) and 6′ sialyllactose (6′SL), 3′fucosyllactose (3-FL), difucosyllactose (DFL), lacto-N-fucopentaose (LNFP such as lacto-N-fucopentaose I, lacto-N-fucopentaose II, lacto-N-fucopentaose III, lacto-NfucopentaoseV), lacto-N-fucohexaose, lacto-N-difucohexaose (LNDFH such as lacto-N-difucohexaose I and lacto-N-difucohexaose II), sialyl-lacto-N-tetraose (LSTa), sialyl-lacto-N-tetraose b (LSTb), sialyl-lacto-N-tetraose c (LSTc) disialyllacto-N-tetraose (DSLNT) Lacto-N-neodifucohexaose (LNnDFH I), fucosyllacto-Nhexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose and tri-fuco-para-Lacto-N-hexaose I, lacto-N-tetraose (LNT), or any combination thereof.
13. Nutritional composition according to the preceding embodiments in addition containing further indigestible carbohydrates, preferably one or more from the group of FOS and GOS carbohydrates.
14. Nutritional composition according to the preceding embodiments obtainable by dry blending HMO particles, and optionally at least part of the digestible carbohydrates and optionally the indigestible carbohydrates, with a base powder that comprises the large lipid globules and at least part of the protein and optionally at least part of the digestible carbohydrates.
15. Nutritional composition according to embodiment 14 wherein the base powder is obtainable by dosing the lipid to an aqueous phase comprising protein and optionally part of the digestible carbohydrates and at least part of the phospholipids, mixing the combined aqueous and lipid phase to obtain an emulsion, and subsequently drying the emulsion, preferably in a spray dryer.
16. Process to prepare the nutritional composition of any one of embodiments 1 to 15 comprising the steps of
a) preparing a base powder wherein the digestible carbohydrates are optionally included in the base powder, added in the dry blending step as a solid or both and
b) dry blending said base powder comprising at least the lipid globules and protein with the HMOs particles comprising at least 2′FL.
17. Process according to embodiment 16 wherein the base powder is made by a) providing an aqueous phase with a dry matter content of 10 wt % to 60 wt % (based on total weight of the aqueous phase), which comprises at least one protein component, b) providing a liquid lipid phase, which comprises at least one lipid and c) mixing the lipid phase with the aqueous phase in a ratio of 5 wt % to 50 wt % using a mixer to provide an oil water emulsion and d) drying the emulsion obtained in step c).
18. Process according to embodiments 16 and 17 wherein further ingredients selected from the group of vitamins, minerals, digestible carbohydrates, and further (indigestible) oligosaccharides are dry blended with the base powder and the HMOs.
19. Product obtainable by the process of any one of embodiments 16 to 18.
20. Nutritional composition according to embodiments 1 to 15 and 19, wherein the powdered nutritional composition is reconstituted with water or other food grade aqueous liquid, to form a ready-to drink liquid.
21. Nutritional composition according to embodiments 1 to 15, 19 and 20 for use in the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving recovery of a gut health problem.
22. Use of milk formula particles to reduce powder segregation in a powdered nutritional composition, said nutritional composition comprising i) milk formula particles comprising lipid, protein, digestible carbohydrates, and (ii) HMO particles, wherein the nutritional composition is selected from an infant, follow-on formula and growing up milk, wherein said nutritional composition is not human milk, wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm and wherein the HMO particles comprise at least 2′fucosyllactose (2′FL).

### DETAILED DESCRIPTION OF THE INVENTION

As used herein, the following terms have the following meanings.

An infant is a child under the age of 12 months.

“Infant formula” or “follow-on formula” or “young child formula” means that it concerns a composition that is artificially made or in other words that it is a synthetic composition (i.e., the synthetic composition is not breast milk). Hence the nutritional composition that is administered is an artificial infant formula or an artificial follow-on formula or an artificial young child formula or a synthetic infant formula or a synthetic follow-on formula or a synthetic young child formula. Infant formula refers to nutritional compositions, artificially made, intended for infants of 0 months to about 4 months to 6 months of age and are intended as a substitute for human milk.

Typically, infant formulae are suitable to be used as sole source of nutrition. Such infant formulae are also known as starter formula.

Follow-on formulae are for infants starting with at 4 months to 6 months of life to 12 months of life and are intended to be supplementary feedings for infants that start weaning on other foods. Infant formulae and follow-on formulae are subject to strict regulations, for example for the EU regulations no. 609/2013 and no. 2016/127.

A young child is a child aged between one and three years, also called a toddler.

Young child formula refers to nutritional compositions, artificially made, intended for infants of 12 months to 36 months, which are intended to be supplementary feedings for infants.

The term “HMO” or “HMOs” refers to human milk oligosaccharide(s). These carbohydrates are highly resistant to enzymatic hydrolysis and have biological functions not directly related to their caloric value. HMOs play a pivotal role in the early development of infants and young children including maturation of the immune system. Human milk comprises many different types of HMOs, over 130 different types have been identified to date. HMOs are based on various combinations of glucose, galactose, sialic acid (N-acetylneuraminic acid), fucose and/or N-acetylglucosamine with most of them having a lactose moiety at their reducing end. Sialic acid and/or fucose, when present, occupy terminal positions at the non-reducing ends. The HMOs can further be divided in acidic (such as charges sialic acid containing oligosaccharides) or neutral (such as fucosylated oligosaccharides). In the context of the present invention lactose is not regarded as an HMO species. HMOs can be manufactured by means known in the art.

A “fucosylated oligosaccharide” is a neutral oligosaccharide having a fucose residue. Examples of fucosylated oligosaccharides are 2′FL (2′-fucosyllactose), 3-FL (3-fucosyllactose), difucosyllactose, lacto-N-fucopentaose (e.g., lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

Fucosyllactose (FL) is a non-digestible oligosaccharide present in human milk. It is not present in bovine milk. It consists of three monosaccharide units, fucose, galactose and glucose linked together. Lactose is a galactose unit linked to a glucose unit via a beta 1,4 linkage. A fucose unit is further linked to a galactose unit of a lactose molecule via an alpha 1,2 linkage (2′-fucosyllactose, 2′-FL, Fucα1-2Galβ1-4Glc) or via an alpha-1,3 linkage to the glucose unit of a lactose (3-Fucosyllactose, 3-FL, Galβ1-4 (Fucα1-3)Glc).

“N-acetylated oligosaccharide(s)” encompass both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

A sialylated oligosaccharide” is a charged sialic acid containing oligosaccharide, i.e. an oligosaccharide having a sialic acid residue. It has an acidic nature. Some examples are 3-SL (3′ sialyllactose) and 6-SL (6′ sialyllactose).

As used herein in the context of powder particle size distributions, the percentile Dx(10) is a maximum particle diameter, below which 10% of the sample volume exists. Dx(50) is a maximum particle diameter, below which 50% of the sample volume exists, and is also known as the median particle size by volume. Similarly, Dx(90) is the maximum particle diameter, below which 90% of the sample volume exists. In the same way, one can determine the percentile Dx(Y), Where Y % of the sample is volume is below.

The particle size distribution of a powder sample may be determined using laser diffraction technology, by means of devices such as the Malvern Mastersizer 3000.

As used herein the Euclidean difference refers to a normalized Euclidean distance statistic to quantify the difference between log-ration particle size distributions a and b (with a and b being different powders). The Euclidean difference is mathematically determined by

$$ND ( a , b ) = \frac{1}{D - 1} \sqrt{{\sum }_{i = 1}^{D} {( {a}_{i} - {b}_{i} )}^{2}} \qquad ( I )$$

Wherein D is the number of particle-size categories (for the Malvern 3000 using default bin size distribution, D=101).

The threshold used as cut-off value that powders have a different distribution used herein is at least, preferably more than 0.2. The higher the Euclidean difference is, the more different the distribution between two powders is.

The nutritional composition according to the present invention is selected from an infant formula, a follow-on formula, and a growing-up milk. This means that the present nutritional composition is not human milk. Alternatively, the term “formula” means that it concerns a composition that is artificially made or in other words that it is synthetic. Hence in one embodiment the nutritional composition is selected from an artificial infant formula, an artificial follow-on formula and an artificial growing-up milk or a synthetic infant formula, a synthetic follow-on formula and a synthetic growing-up milk.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

### Formula

The nutritional composition according to the invention is in the form an infant formula, a follow-on formula, or a young child formula. This means that the composition that is to be administered is not human milk. It also means that the nutritional composition is not native cow's milk or native milk from another mammal. In the context of the present invention, young child formula can also be named growing-up milk.

Alternatively, the terms as used herein, “infant formula” or “follow-on formula” or “young child formula” means that it concerns a composition that is artificially made or in other words that it is synthetic. Hence in one embodiment, the nutritional composition that is administered is an artificial infant formula or an artificial follow-on formula or an artificial young child formula or a synthetic infant formula or a synthetic follow-on formula or a synthetic young child formula.

In the present invention, infant formula refers to nutritional compositions, artificially made, intended for infants of 0 months to about 4 months to 6 months of age and are intended as a substitute for human milk. Typically, infant formulae are suitable to be used as sole source of nutrition. Such infant formulae are also known as starter formula. Follow-on formula for infants starting with at 4 months to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants.

The nutritional composition is preferably an infant formula or a follow-on formula. More preferably the nutritional composition is an infant formula.

The nutritional composition comprises digestible carbohydrates, protein, and lipid, wherein the lipid preferably provides 30 to 60% of the total calories, the protein provides 5% to 20% of the total calories and the digestible carbohydrates provide 25% to 75% of the total calories.

The nutritional composition is preferably an infant formula or follow-on formula and preferably comprises 3 g to 7 g lipid/100 kcal, preferably 4 g to 6 g lipid/100 kcal, more preferably 4.5 g to 5.5 g lipid/100 kcal, preferably comprises 1.7 g to 3.5 g protein/100 kcal, more preferably 1.8 g to 2.1 g protein/100 kcal, more preferably 1.8 g to 2.0 g protein/100 kcal and preferably comprises 5 g to 20 g digestible carbohydrate/100 kcal, preferably 6 g to 16 g digestible carbohydrate/100 kcal, more preferably 10 g to 15 g digestible carbohydrate/100 kcal.

Preferably the nutritional composition is an infant formula or follow-on formula, and preferably has an energy density of 60 kcal/100 ml to 75 kcal/100 ml, more preferably 60 kcal/100 ml to 70 kcal/100 ml, when in a ready-to-drink form. This density ensures an optimal balance between hydration and caloric intake.

The nutritional composition is a solid product, preferably a powder. Suitably, the nutritional composition is in a powdered form, which can be reconstituted with water or other food grade aqueous liquid, to form a ready-to drink liquid. It was found that lipid globules maintained their size and coating when reconstituted. It was further found that the nutritional composition according to the invention with milk formula particles and HMO particles did not suffer from segregation.

The nutritional compositions according to the present invention comprises milk formula particles comprising carbohydrates, protein, and lipids wherein preferably the lipids provide 30 to 60% of the total calories, the protein provides 5 to 20% of the total calories and the carbohydrates provide 25 to 75% of the total calories. Preferably, the nutritional composition comprises 10 to 50 wt. % lipids based on dry weight of the total composition.

The powdered nutritional composition preferably has a dry matter content of at least 90 wt. %, more preferably 92.5-100 wt. %, even more preferably 95-99.9 wt. %.

Preferably, the powdered nutritional composition comprises 85-99.5 wt. % of milk formula particles, more preferably 90-99 wt. % of milk formula particles.

Preferably, the powdered nutritional composition comprises 0.1-3 wt. % of HMO particles, more preferably 0.15-2.5 wt. % of HMO particles.

The weight ratio between the milk formula particles and the HMO particles in the powdered nutritional composition is preferably at least 10:1, more preferably 25:1-1500:1, and most preferably 50:1-1000:1.

Preferably, the particle size distribution of the milk formula particles comprises a Dx(10) of 50 μm to 110 μm, preferably 50 μm to 90 μm, more preferably 60 μm to 80 μm, even more preferably 65 μm to 75 μm. In an embodiment the milk formula particles comprise a Dx(10) of at least 50 μm, more preferably at least 60 μm, even more preferably at least 70 μm. The volume of the particles and its size distribution can suitably be determined using a particle size analyser such as a Mastersizer 3000 (Malvern Instruments, Malvern, UK).

Preferably, the particle size distribution of the milk formula particles comprises a Dx(20) of 115 μm to 175 μm, preferably 115 μm to 145 μm, more preferably 120 μm to 140 μm, even more preferably 125 μm to 135 μm. In an embodiment the milk formula particles comprise a Dx(20) of at least 115 μm, more preferably at least 120 μm, even more preferably at least 125 μm.

Preferably the particle size distribution of the milk formula particles comprises a Dx(50) of 200 μm to 300 μm, preferably 200 μm to 260 μm, more preferably 210 μm to 250 μm, even more preferably 220 μm to 240 μm. In an embodiment the milk formula particles comprise a Dx(50) of at least 200 μm, more preferably at least 210 μm, even more preferably at least 220 μm.

Preferably, the particle size distribution of the milk formula particles comprises a Dx(80) of 330 μm to 450 μm, preferably 330 μm to 370 μm, more preferably 340 μm to 360 μm, even more preferably 345 μm to 355 μm. In an embodiment the milk formula particles comprise a Dx(80) of at least 330 μm, more preferably at least 340 μm, even more preferably at least 345 μm.

Preferably the particle size distribution of the milk formula particles comprises a Dx(90) of 400 μm to 550 μm, preferably 400 μm to 460 μm, more preferably 410 μm to 450 μm, even more preferably 420 μm to 440 μm. In an embodiment the milk formula particles comprise a Dx(90) of at least 400 μm, more preferably at least 410 μm, even more preferably at least 420 μm.

Preferably, the particle size distribution of the milk formula particles comprises a Dx(10) to Dx(90) range of from 50 μm to 550 μm, preferably of from 50 μm to 460 μm, more preferably of from 60 μm to 450 μm, even more preferably of from 70 μm to 440 μm. As used herein the Dx(10) is the diameter at which 10% of the composition's volume is comprised of particles with a diameter less than this value while Dx(90) is the diameter at which 90% of the composition's volume is comprised of particles with a diameter less than this value; the Dx(10) to Dx(90) range as used herein is the diameter range at which 80% of the powder's volume is comprised of particles with a diameter in the range. Likewise the Dx(20) to Dx(80) range is the diameter range at which 60% of the powder's volume is comprised of particles with a diameter in said range.

Preferably, the particle size distribution of the HMO particles comprises a Dx(10) of 10 μm to 60 μm, more preferably 15 μm to 55 μm, even more preferably 20 μm to 50 μm. In an embodiment the milk formula particles comprise a Dx(10) of at least 5 μm, more preferably at least 10 μm, even more preferably at least 15 μm.

Preferably, the particle size distribution of the HMO particles comprises a Dx(20) of 20 μm to 80 μm, more preferably 25 μm to 75 μm, even more preferably 30 μm to 70 μm. In an embodiment the milk formula particles comprise a Dx(20) of at least 20 μm, more preferably at least 25 μm, even more preferably at least 30 μm.

Preferably, the particle size distribution of the HMO particles comprises a Dx(50) of 40 μm to 130 μm, more preferably 45 μm to 125 μm, even more preferably 50 μm to 120 μm. In an embodiment the milk formula particles comprise a Dx(50) of at least 40 μm, more preferably at least 45 μm, even more preferably at least 50 μm.

Preferably, the particle size distribution of the HMO particles comprises a Dx(80) of 65 μm to 220 μm, more preferably 70 μm to 215 μm, even more preferably 75 μm to 210 μm. In an embodiment the milk formula particles comprise a Dx(80) of at least 65 μm, more preferably at least 70 μm, even more preferably at least 75 μm.

Preferably, the particle size distribution of the HMO particles comprises a Dx(90) of 80 μm to 315 μm, more preferably 85 μm to 310 μm, even more preferably 90 μm to 305 μm. In an embodiment the milk formula particles comprise a Dx(90) of at least 80 μm, more preferably at least 85 μm, even more preferably at least 90 μm.

Preferably the particle size distribution of the HMO particles comprises a Dx(10) to Dx(90) range of from 5 μm to 315 μm, more preferably of from 10 μm to 310, even more preferably of from 15 μm to 305 μm.

In a preferred embodiment the difference between the Dx(20) of the milk formula particles and the Dx(80) of the HMO particles is at least 10 μm, more preferably at least 15 μm, even more preferably at least 20 μm. Worded differently, the Dx(20) of the milk formula particles is at least 10 μm higher than the Dx(80) of the HMO particles, more preferably at least 15 μm, even more preferably at least 20 μm. The latter thus means that at a certain Dx(80) value of the HMO particles the Dx(20) of the milk formula particles is at least 10 μm larger or higher, e.g. at a Dx(80) of the HMO particles of 80 μm, the Dx(20) of the milk formula particles is at least 10 μm larger, i.e. at least 90 μm.

In a preferred aspect the Dx(20) of the milk formula particles is at least 5 μm higher than the Dx(50) of the HMO particles, more preferably at least 7.5 μm, even more preferably at least 10 μm. In some embodiments the Dx(20) of the milk formula particles is at least 50 μm higher than the Dx(50) of the HMO particles. Worded differently, preferably the difference between the Dx(20) of the milk formula particles and the Dx(50) of the HMO particles is at least 5 μm, more preferably at least 7.5 μm, even more preferably at least 10 μm.

Preferably, the Dx(50) of the milk formula particles is at least 2.5 times the Dx(50) of the HMO particles. More preferably, the Dx(50) of the milk formula particles is at least 3 times the Dx(50) of the HMO particles. Most preferably, the Dx(50) of the milk formula particles is at least 4 times the Dx(50) of the HMO particles.

In a further aspect, preferably, the Dx(10) of the milk formula particles is at least 2 times the Dx(10) of the HMO particles. More preferably, the Dx(10) of the milk formula particles is at least 2.5 times the Dx(10) of the HMO particles, even more preferably the Dx(10) of the milk formula particles is at least 3 times the Dx(10) of the HMO particles. Most preferably, the Dx(50) of the milk formula particles is at least 3.5 times the Dx(50) of the HMO particles.

Preferably, the Dx(90) of the milk formula particles is at least 2.5 times the Dx(50) of the HMO particles. More preferably, the Dx(90) of the milk formula particles is at least 3 times the Dx(90) of the HMO particles. Most preferably, the Dx(50) of the milk formula particles is at least 4 times the Dx(90) of the HMO particles.

The Euclidean difference between the milk formula particles and the HMO particles is preferably more than 0.2, more preferably at least 0.25, even more preferably at least 0.3. The Euclidean difference as used herein provides a quantitative measure of the difference in the particle size distribution between powders.

### HMOs

Human milk contains an abundance of structurally diverse oligosaccharides, known collectively as human milk oligosaccharides (HMOs), which support immune function through several ways. HMOs are thus (short) polymers of sugars (oligosaccharides) like the ones in human breast milk able to promote the development of the immune system, to reduce the pathogen infections and to improve brain development and cognition. In addition, HMOs are considered to shape the gut microbiota of an infant by selectively stimulating bacteria (Bode et al, Human milk oligosaccharides: every baby needs a sugar mama. Glycobiology 2012; 22(9): 1147-1162). The HMOs as used herein are preferably in the form of HMO particles.

The nutritional compositions according to the invention comprise at least one type of HMOs that is 2′-fucosyllactose (2′FL).

In an embodiment the HMOs comprised in the nutritional composition preferably comprise at least 40 wt %, more preferably at least 45 wt %, even more preferably at least 50 wt % of 2′FL based on total HMO weight.

In an alternative preferred embodiment, the HMOs comprise 90-100 wt % of 2′FL, even more preferably 95 wt. %-100 wt % of 2′FL based on total HMOs weight.

Fucosylated oligosaccharide(s) may be isolated by chromatography or filtration technology from a natural source such as animal milks. Alternatively, it may be produced by biotechnological means using specific fucosyltransferases and/or fucosidases either using enzyme-based fermentation technology (recombinant or natural enzymes) or microbial fermentation technology known in the art. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Alternatively, fucosylated oligosaccharides may be produced by Chemical synthesis from lactose and free fucose. Fucosylated oligosaccharides are also available for example from Kyowa, Hakko, Kogyo of Japan, Friesland Campina, The Netherlands, Glycom DSM, Denmark and Chr. Hansen, Denmark.

In an embodiment the HMOs comprised in the nutritional composition may in addition to 2′FL also comprise at least another oligosaccharide(s), preferably an N-acetylated oligosaccharide. There can be one or more types of N-acetylated oligosaccharide. The N-acetylated oligosaccharide(s) may be selected from lacto-N-tetraose (LNT), lacto-N-neotetraose (LNnT) or any combination thereof. In some particular embodiments the N-acetylated oligosaccharide is lacto-N-neotetraose (LNnT), para-lacto-N-neohexaose (para-LNnH) or any combination thereof.

In an embodiment the N-acetylated oligosaccharide is LNnT. In an embodiment the HMOs comprised in the nutritional composition preferably comprise 10-40 wt % LNnT, more preferably 15-35 wt % LNnT, even more preferably 20-30 wt % LNnT based on total weight of the HMOs.

In a preferred embodiment the N-acetylated oligosaccharide is LNT. In an embodiment the HMOs preferably comprise 10-40 wt % LNT, more preferably 15-35 wt % LNT, even more preferably 20-30 wt % LNT based on total weight of the HMOs. In a particularly advantageous embodiment of the present invention, the HMOs comprised in the nutritional composition comprises 2′-fucosyllactose (2FL) and lacto-N-neotetraose (LNnT).

In some other particular embodiments, the N-acetylated oligosaccharide is a mixture of LNT and LNnT. In some particular embodiments the composition comprises both LNT and LNnT in a ratio LNT: LNnT between 5:1 and 1:2, or from 2:1 to 1:1, or from 2:1.2 to 2:1.6.

The N-acetylated oligosaccharide(s) may be synthetized by enzymatic transfer of saccharide units using glycosyltransferases as described in U.S. Pat. No. 5,288,637 or by other conversion methods known in the art. N-acetylated oligosaccharides suitable for the preparation of and for use in the nutritional composition according to the invention include commercially available variants such as those from Glycom DSM, Denmark and Chr. Hansen, Denmark.

In a particular embodiment, the HMOs of the nutritional composition according to the invention can comprise sialylated oligosaccharide(s). There can be one or several sialylated oligosaccharide(s).

The sialylated oligosaccharide(s) can be selected from the group comprising 3′ sialyllactose (3′SL), 6′ sialyllactose (6′SL), and any combination thereof. In some embodiments of the invention the composition comprises 3′SL and 6′SL. In some particular embodiments the ratio between 3′-sialyllactose (3′SL) and 6′-sialyllactose (6′SL) can be in the range between 5:1 and 1:10, or from 3:1 and 1:1, or from 1:1 to 1:10.

The composition according to the present invention may optionally also comprise at least one precursor of oligosaccharides. There can be one or several precursor(s) of oligosaccharide. For example, the precursor of human milk oligosaccharide is sialic acid, fucose or a mixture thereof. In some particular embodiments the composition comprises sialic acid.

In another embodiment the HMOs comprise 2′FL and one or more further HMOs components. The HMO component comprises 2′FL and may comprise at least one, preferably more selected from sialic acid (SA), lacto-N-neotetraose (LNnT), para-lacto-N-neohexaose (para-LNnH), sialic acid, 3′ sialyllactose (3′SL) and 6′ sialyllactose (6′SL), 3′fucosyllactose (3-FL), difucosyllactose (DFL), lacto-N-fucopentaose (LNFP such as lacto-N-fucopentaose I, lacto-N-fucopentaose II, lacto-N-fucopentaose III, lacto-N-fucopentaoseV), lacto-N-fucohexaose, lacto-N-difucohexaose (LNDFH such as lacto-N-difucohexaose I and lacto-N-difucohexaose II), sialyl-lacto-N-tetraose (LSTa), sialyl-lacto-N-tetraose b (LSTb), sialyl-lacto-N-tetraose c (LSTc), disialyllacto-N-tetraose (DSLNT) Lacto-N-neodifucohexaose (LNnDFH I), fucosyllacto-Nhexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose and tri-fuco-para-Lacto-N-hexaose I, and lacto-N-tetraose (LNT) or any combination thereof. In some particular embodiments the HMO component comprises 2′FL and one or more from 3′fucosyllactose (3-FL), lacto-N-tetraose (LNT), lacto-N-neotetraose (LNnT), difucosyllactose (DFL), 3′ sialyllactose (3′SL) and 6′ sialyllactose (6′SL).

Exemplary HMO combinations include but are not limited to: SA, 3′SL, 6′SL, 3-FL, 2′FL, and LNnT; SA, 3′SL, 6′SL, 3-FL, 2′FL, LNT and LNnT; SA, 3′SL, 6′SL, 3-FL, 2′FL, DFL, LNT and LNnT; 3′SL, 6′SL, 3-FL, 2′FL, DFL, LNT and LNnT; 3′SL, 6′SL, 3-FL, 2′FL, and LNnT; 3′SL, 6′SL, 3-FL, 2′FL, and LNT; 3′SL, 6′SL, 3-FL, 2′FL, and DFL; 3′SL, 6′SL, 2′FL, LNT and DFL; SA, 6′SL, 3-FL, 2′FL, and LNnT; SA, 6′SL, 3-FL, 2′FL, and LNT; SA, 6′SL, 3-FL, 2′FL, and DFL; SA, 3′SL, 3-FL, 2′FL, and LNnT; SA, 3′SL, 6′SL, 2′FL, and LNnT; SA, 3′SL, 6′SL, 2′FL, and LNT; SA, 3′SL, 6′SL, 2′FL, and DFL; SA, 3′SL, 6′SL, 3-FL, and 2′FL; SA and 2′FL; 3′SL and 2′FL; SA, 6′SL, and 2′FL; SA, 3-FL, and 2′FL; SA, 2′FL, and LNnT; 2′FL, and LNT, 2′FL, and DFL, SA, 3′SL, 6′SL and 2′FL; SA, 3′SL, 3-FL, and 2′FL; SA, 3′SL, 2′FL, and LNnT; SA, 6′SL, 3-FL, and 2′FL; SA, 6′SL, 2′FL, and LNnT; SA, 3-FL, 2′FL, and LNnT; SA, 6′SL, 2′FL, and LNnT; SA, 3′SL, 3-FL, 2′FL, and LNnT; SA, 6′SL, 3-FL, 2′FL, and LNnT; SA, 3′SL, 3-FL, 2′FL, and LNnT; SA, 3′SL, 6′SL, 2′FL, and LNnT; 3′SL, 6′SL, 3-FL, and 2′FL; 3′SL, 6′SL, 2′FL, and LNnT; 3′SL, 3-FL, 2′FL, and LNnT; 3′SL, 3-FL, and 2′FL; 3′SL, 2′FL, and LNnT; 3′SL, 2′FL, and LNT; 3′SL, 2′FL, and DFL; 3′SL, 6′SL, and 2′FL; 3′SL and 3-FL; 3′SL and 2′FL; 6′SL and 2′FL; 6′SL, 3-FL, 2′FL, and LNnT; 3-FL, 2′FL, and LNnT; 2′FL and LNT; 2′FL and DFL and 2′FL and LNnT.

In a preferred aspect the HMOs in addition to 2′FL may be selected from 3-FL (3-Fucosyllactose). 6′SL (6-sialyllactose), 3′SL (3′-sialyllactose), LNT (lacto-N-tetraose), LNnT (lacto-N-neotetraose) and difucosyllactose (DFL).

In a preferred embodiment the HMOs comprise 40-60 wt % 2′FL, 10-20 wt % 3-FL, 2-7% 3′SL, 4-8% 6′SL and 20-30% LNT based on total weight of the HMOs.

In an embodiment wherein 2′-fucosyllactose (2′FL) and 3′-fucosyllactose (3-FL) are both incorporated in the composition they are preferably used in a weight ratio 2′FL:3-FL of at least 2.5:1. When 3′-sialyllactose (3′SL) is incorporated in the composition it is preferably in a weight ratio 2′FL:3′SL of at least 11.2:1. When 6′-sialyllactose (6′SL) is included in the composition it is preferably in a weight ratio 2′FL:6′SL of at least 1.8:1.

In a preferred embodiment of the invention, the HMOs may be present in an amount of 300-2500 μg/ml of the reconstituted powder composition, more preferably 400-2200 μg/ml, even more preferably 750-2000 μg/ml of the composition. In a particular embodiment, the HMO mix is in an amount of 1500-2000 μg/ml of the composition. Such amounts are particularly adequate for complete nutrition such as an infant formula, follow-on formula or in the case of a growing-up milk.

In case wherein the nutritional composition is in powder form, the HMOs may preferably be present in an amount in the range of 300 mg/100 g dry weight-1400 mg/100 g dry weight, preferably in the range of 450 mg/100 g dry weight-1250 mg/100 g dry weight. When expressed in amounts based on calories, preferably the nutritional composition according to the invention comprises HMOs in a concentration in the range of 60 mg/100 kcal-300 mg/100 kcal, preferably in the range of 100 mg/100 kcal-270 mg/100 kcal.

In one embodiment, the HMOs are provided in the nutritional composition of the present invention in such an amount that normal consumption of the nutritional composition would provide to the infant or young child, respectively the child, consuming it a total daily dose of 0.1 to 10 g, such as 0.2-9 g, 0.3-8 g, 0.4-7 g, 0.5-6 g, 0.6-5 g, 0, 8-3 g, 0.9-2 g or 1 to 1.5 g per day.

Preferably, the nutritional composition according to the invention comprises, when reconstituted, 2′FL in a concentration in the range of 200 μg/ml-1500 μg/ml, more preferably in the range of 250 μg/ml-1250 μg/ml.

When expressed in amounts based on dry weight, preferably the nutritional composition according to the invention comprises 2′FL in a concentration in the range of 175 mg/100 g dry weight-1125 mg/100 g dry weight, preferably in the range of 560 mg/100 g dry weight-940 mg/100 g dry weight. When expressed in amounts based on calories, preferably the nutritional composition according to the invention comprises 2′FL in a concentration in the range of 35 mg/100 kcal-225 mg/100 kcal, preferably in the range of 50 mg/100 kcal-190 mg/100 kcal.

As indicated for the individual HMOs, the HMOs suitable for the preparation of the nutritional composition according to the present invention are commercially available, for example from Kyowa, Hakko, Kogyo of Japan, Friesland Campina, The Netherlands, Glycom DSM, Denmark and Chr. Hansen, Denmark. Otherwise, it is well within the reach of the skilled person to obtain the HMOs by isolation from suitable sources or by chemical synthesis using methods known in the art.

Base Powder with Large Fat Droplets

### Lipid

The nutritional composition for use according to the present invention comprises milk powder particles comprising lipid that is present in globules as defined above.

Lipid in the present invention comprises one or more selected from the group consisting of triglycerides, polar lipids (such as phospholipids, cholesterol, glycolipids, sphingomyelin), free fatty acids, monoglycerides and diglycerides. Preferably the composition comprises at least 70 wt. %, more preferably at least 80 wt. %, even more preferably at least 85 wt. % triglycerides, most preferably at least 90 wt. % triglycerides based on total lipid.

The lipid provides preferably 30% to 60% of the total calories of the nutritional composition. More preferably the nutritional composition comprises lipid providing 35% to 55% of the total calories, even more preferably the nutritional composition comprises lipid providing 40 to 50% of the total calories. The lipid is preferably present in an amount of 3 g to 7 g per 100 kcal, more preferably in an amount of 4 g to 6 g lipid per 100 kcal and most preferably in an amount of 4.5 g to 5.5 g lipid per 100 kcal. When in liquid form, e.g., as a reconstituted ready-to-feed liquid, the nutritional composition preferably comprises 2.1 g to 6.5 g lipid per 100 ml, more preferably 3.0 g to 4.0 g per 100 ml. Based on dry weight the nutritional composition preferably comprises 10 wt % to 50 wt %, more preferably 12.5 wt % to 40 wt % lipid, even more preferably 19 wt % to 30 wt % lipid. Worded alternatively, when the nutritional composition is in powder form, the lipids are preferably present in an amount of 10 g-50 g/100 g dry weight, more preferably 12.5 g-40 g/100 g dry weight, even more preferably 19 g-30 g/100 g dry weight of the composition.

The lipid preferably comprises vegetable lipid. The presence of vegetable lipid advantageously enables an optimal fatty acid profile high in polyunsaturated fatty acids and/or more reminiscent to human milk fat. Lipid from non-human mammalian milk alone, e.g. cow milk, does not provide an optimal fatty acid profile. The amount of essential fatty acids is too low in non-human mammalian milk.

Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

In a preferred embodiment, the nutritional composition comprises 30 wt % to 90 wt % vegetable lipids based on total lipid, more preferably 35 wt % to 80 wt %, more preferably 40 wt % to 70 wt %, more preferably 40 wt % to 60 wt % vegetable lipids based on total lipid.

The lipid in the nutritional composition preferably further comprises mammalian milk fat, preferably ruminants milk fat, more preferably the mammalian milk fat is derived from cow milk, goat milk, sheep milk, buffalo milk, yak milk, reindeer milk, and/or camel milk, most preferably the mammalian milk fat is cow milk fat. Preferably the mammalian milk fat is not human milk fat. Preferably the mammalian milk fat comprises at least 70 wt % triglycerides, more preferably at least 90 wt %, more preferably at least 97 wt % triglycerides by weight of the mammalian milk fat.

Preferably the mammalian milk fat is derived from butter, butter fat, butter oil, and/or anhydrous milk fat, more preferably the mammalian milk fat is derived from anhydrous milk fat and/or butter oil. Such mammalian milk fat sources are high in triglyceride levels. These mammalian milk fat sources may be in the form of a continuous lipid phase or a water-in-oil emulsion. The use of these mammalian milk fat sources during the manufacture of the nutritional composition of the present invention enables the formation of lipid globules, wherein each globule comprises a mixture of vegetable fat and mammalian milk fat.

Mammalian milk fat in the present invention refers to all lipid components of milk, as produced by the mammalians, such as the cow, and is found in commercial milk and milk-derived products. Butter in the present invention is a water-in-oil emulsion comprised of over 80 wt % milk fat. Butterfat in the present invention relates to all of the fat components in milk that are separable by churning, in other words, present in butter. Anhydrous milk fat (AMF) is a term known in the art and relates to extracted milk fat. Typically, AMF comprises more than 99 wt % lipid based on total weight. It can be prepared from extracting milk fat from cream or butter. Anhydrous butter oil in the present invention is synonymous with AMF. Butteroil also is a term known in the art. It typically relates to a milk fat extract with more than 98 wt % lipid and typically is a precursor in the process of preparing anhydrous milk fat or anhydrous butter oil.

Preferably the composition comprises 10 wt % to 70 wt % mammalian milk fat based on total lipid, more preferably 20 wt % to 65 wt %, more preferably 30 wt % to 60 wt %, more preferably 40 wt % to 60 wt % based on total lipid.

Preferably the ratio of vegetable fat to mammalian milk fat ranges from 3/7 to 9/1. Alternatively worded, in a preferred embodiment the lipid contains both vegetable fat and milk fat, preferably in a weight ratio of between 30:70 and 90:10.

In a preferred embodiment, the lipid in the nutritional composition comprises:
a) 35 wt % to 80 wt % vegetable lipid based on total lipid, and
b) 20 wt % to 65 wt % mammalian milk fat based on total lipid, wherein the mammalian milk fat is selected from butter, butter fat, butter oil or anhydrous milk fat.

More preferably, the lipid in the nutritional composition comprises:
a) 40 wt % to 70 wt % vegetable lipid based on total lipid, and
b) 30 wt % to 60 wt % mammalian milk fat based on total lipid, wherein the mammalian milk fat is selected from butter, butter fat, butter oil or anhydrous milk fat.

Most preferably, the lipid in the nutritional composition comprises:
a) 40 wt % to 60 wt % vegetable lipid based on total lipid, and
b) 40 wt % to 60 wt % mammalian milk fat based on total lipid, wherein the mammalian milk fat is selected from butter, butter fat, butter oil or anhydrous milk fat.

The nutritional composition preferably also comprises one or more of fish oil, egg lipid, and microbial, algal, fungal, or single cell oils.

Compared to vegetable fat, mammalian milk fat is known to have a higher content of palmitic acid (PA) at the sn-2 position of a triglyceride. In a preferred embodiment, the lipid in the nutritional composition comprises at least 10 wt % PA based on total fatty acids and at least 15 wt % of PA, based on total palmitic acid, is located at the sn-2 position of a triglyceride. Preferably, the amount of PA is below 30 wt % based on total fatty acids. More preferably, the amount of PA is from 12 wt % to 26 wt % based on total fatty acids, even more preferably from 14 wt % to 24 wt. %.

Preferably, at least 15 wt % PA, more preferably at least 20 wt % PA, even more preferably at least 25 wt % PA, most preferably at least 30 wt % PA, based on total PA is in the sn-2 or beta position in a triglyceride. Preferably the amount of PA in the sn-2 position in a triglyceride is not more than 45 wt %, preferably not more than 40 wt % based on total PA present in the lipid. Preferably the amount of PA in the sn-2 position in a triglyceride is from 25 wt % to 40 wt % based on total PA.

Compared to vegetable fat, mammalian milk fat is known to have a higher content of short-chain fatty acids (SCFA) butyric acid (BA; C4:0) and caproic acid (CA; C6:0). In a preferred embodiment, the lipid in the nutritional composition comprises 0.6 wt % to 5 wt % SCFA being the sum of BA and CA based on total fatty acids. Preferably the nutritional composition comprises less than 5 wt % BA based on total fatty acids, preferably less than 4 wt %. Preferably the nutritional composition comprises at least 0.5 wt % butyric acid based on total fatty acids, preferably at least 0.6 wt %, preferably at least 0.9 wt %, more preferably at least 1.2 wt % BA based on total fatty acids.

In a preferred embodiment, the lipid in the nutritional composition comprises:
at least 10 wt % PA based on total fatty acids and at least 15 wt % of PA, based on total PA, is located at the sn-2 position of a triglyceride; and.
0.6 wt % to 5 wt % SCFA being the sum of BA and CA based on total fatty acids.

### Fatty Acid Composition

SFA relates to saturated fatty acids and/or acyl chains, MUFA relates to mono-unsaturated fatty acid and/or acyl chains, PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds; Medium chain fatty acids (MCFA) refer to fatty acids and/or acyl chains with a chain length of 6, 8 or 10 carbon atoms. n3 or omega-3 PUFA refers to polyunsaturated fatty acids and/or acyl chains with 2 or more unsaturated bonds with an unsaturated bond at the third carbon atom from the methyl end of the fatty acyl chain, n6 or omega-6 PUFA refers to polyunsaturated fatty acids and/or acyl chains with 2 or more unsaturated bonds with an unsaturated bond at the sixth carbon atom from the methyl end of the fatty acyl chain.

In the context of the present invention, a weight percentage of fatty acids based on total fatty acids is calculated as if all fatty acids are free fatty acids, hence it is not taken into account whether a fatty acid is attached to a glycerol backbone or not.

DHA refers to docosahexaenoic acid and/or acyl chain (22:6 n3); DPA refers to docosapentaenoic acid and/or acyl chain (22:5 n3); n6 DPA refers to omega-6 docosapentaenoic acid and/or acyl chain (22:5 n6). EPA refers to eicosapentaenoic acid and/or acyl chain (20:5 n3); ARA refers to arachidonic acid and/or acyl chain (20:4 n6). LA refers to linoleic acid and/or acyl chain (18:2 n6); ALA refers to alpha-linolenic acid and/or acyl chain (18:3 n3). PA relates to palmitic acid and/or acyl chains (C16:0). BA refers to butyric acid (C4:0). CA refers to caproic acid (C6:0).

LA refers to linoleic acid and/or acyl chain and is an n6 PUFA (18:2 n6) and the precursor of n6 LC-PUFA and is an essential fatty acid as it cannot be synthesized by the human body. The nutritional composition preferably comprises LA. LA preferably is present in a sufficient amount to promote a healthy growth and development, yet in an amount as low as possible to prevent negative, competitive, effects on the formation of n3 PUFA and a too high n6/n3 ratio. The nutritional composition therefore preferably comprises less than 20 wt % LA based on total fatty acids, preferably 5 wt % to 16 wt %, more preferably 10 wt % to 14.5 wt %. Preferably, the nutritional composition comprises at least 5 wt % LA based on total fatty acids, preferably at least 6 wt % LA, more preferably at least 7 wt % LA based on total fatty acids. Per 100 kcal, the nutritional composition preferably comprises 350 mg-1400 mg LA.

ALA refers to alpha-linolenic acid and/or acyl chain and is an n3 PUFA (18:3 n3) and the precursor of n3 LC-PUFA and is an essential fatty acid as it cannot be synthesized by the human body. The nutritional composition preferably comprises ALA. Preferably ALA is present in a sufficient amount to promote a healthy growth and development of the infant. The nutritional composition preferably comprises at least 1.0 wt. %, more preferably the nutritional composition comprises at least 1.5 wt. %, even more preferably at least 2.0 wt. % ALA based on total fatty acids. Preferably the nutritional composition comprises less than 10 wt. % ALA, more preferably less than 5.0 wt. %, based on total fatty acids.

Preferably the nutritional composition comprises a weight ratio of LA/ALA from 2 to 20, more preferably from 3 to 16, even more preferably from 4 to 14, most preferably from 5 to 12.

The lipid in the nutritional composition preferably comprises 5 to 35 wt. % PUFA, based on total fatty acids, comprising LA and ALA in a weight ratio LA/ALA of 2 to 20.

Preferably, the nutritional composition comprises n3 LC-PUFA, such as EPA, DPA and/or DHA, more preferably DHA. As the conversion of ALA to DHA may be less efficient in infants, preferably both ALA and DHA are present in the nutritional composition. Preferably the nutritional composition comprises at least 0.05 wt. %, preferably at least 0.1 wt. %, more preferably at least 0.2 wt. %, of DHA based on total fatty acids. Preferably the nutritional composition comprises not more than 2.0 wt. %, preferably not more than 1.0 wt. % of DHA based on total fatty acids.

The nutritional composition preferably comprises ARA. Preferably the nutritional composition comprises at least 0.05 wt. %, preferably at least 0.1 wt. %, more preferably at least 0.2 wt. % of ARA based on total fatty acids. As the group of n6 fatty acids, especially arachidonic acid (ARA) counteracts the group of n3 fatty acids, especially DHA, the nutritional composition preferably comprises relatively low amounts of ARA. Preferably the nutritional composition comprises not more than 2.0 wt. %, preferably not more than 1.0 wt. % of ARA based on total fatty acids. Preferably the weight ratio between DHA and ARA is between 1:4 to 4:1, more preferably between 1:2 to 2:1, more preferably between 0.6 and 1.5.

### Lipid Globule Size

The lipid is present in the milk formula particles in the form of lipid globules. The lipid globules comprise a core and a surface.

The powdered nutritional composition thus comprises lipid globules. The lipid in the nutritional composition is in the form of lipid globules and wherein:
a. the lipid globules have a mode diameter, based on volume, of at least 1.0 μm; and/or
b. at least 40 volume %, based on total lipid volume, of the lipid globules have a diameter of 2 to 12 μm.

Lipid is typically present in the milk formula particles of the nutritional composition in the form of lipid globules. When the powdered nutritional composition is reconstituted in liquid form, these lipid globules are emulsified in the aqueous phase. Alternatively, when the nutritional composition is in powder form, the lipid globules are present in the milk formula powder and the powder is suitable for reconstitution with water or another food grade aqueous phase. The lipid globules comprise a core and a surface.

The core preferably comprises vegetable lipid. The core preferably comprises at least 90 wt. % triglycerides and more preferably essentially consists of triglycerides. Not all vegetable lipids that are present in the composition need necessarily be comprised in the core of lipid globules, but preferably a major part is, preferably more than 50% wt. %, more preferably more than 70 wt. %, even more preferably more than 85 wt. %, even more preferably more than 95 wt. %, most preferably more than 98 wt. % of the vegetable lipids that are present in the composition are comprised in the core of lipid globules. In one embodiment the core of the lipid globules comprises at least 40 wt. % triglycerides of vegetable origin, more preferably at least 50 wt. %, even more preferably at least 70 wt. % triglycerides of vegetable origin, more preferably the core of the lipid globules comprises at least 85 wt. %, more preferably at least 95 wt. % triglycerides of vegetable origin.

The lipid globules in the milk formula particles of the nutritional composition preferably have a mode diameter, based on volume, of at least 1.0 μm, more preferably at least 2.0 μm, and most preferably at least 3.0 μm. Preferably, the lipid globules have a mode diameter, based on volume, between 1.0 and 10 μm, more preferably between 2.0 and 8.0 μm, even more preferably between 3.0 and 7.0 μm, and most preferably between 3.0 μm and 6.0 μm.

Alternatively, or preferably in addition, the size distribution of the lipid globules is preferably in such a way that at least 45 volume % (vol. %), preferably at least 55 vol. %, even more preferably at least 65 vol. %, and most preferably at least 75 vol. % of the lipid globules have a diameter between 2 and 12 μm. In a more preferred embodiment, at least 45 vol. %, preferably at least 55 vol. %, more preferably at least 65 vol. %, and most preferably at least 75 vol. % of the lipid globules have a diameter between 2 and 10 μm. In an even more preferred embodiment, at least 45 vol. %, more preferably at least 55 vol. %, yet even more preferably at least 65 vol. %, and most preferably at least 75 vol. % of the lipid globules have a diameter between 4 and 10 μm. Preferably less than 5 vol. % of the lipid globules have a diameter above 12 μm.

Standard infant formulae, follow-on formulae or young child formulae typically have lipid globules with a mode diameter, based on volume, of about 0.3-0.5 μm and/or less than 45 vol. % of the lipid globules have a diameter above 2 μm.

The volume percentage of lipid globules is based on volume of total lipid. The mode diameter relates to the diameter which is the most present based on volume % of total lipid, or the peak value in a graphic representation, having on the X—as the diameter and on the Y—as the volume %.

The volume of the lipid globule and its size distribution can suitably be determined using a particle size analyzer such as a Mastersizer 2000 (Malvern Instruments, Malvern, UK), for example by the method described in Michalski et al, 2001, Lait 81:787-796.

### Phospholipid

The nutritional composition preferably comprises phospholipids, more preferably phospholipids derived from mammalian milk, even more preferably derived from non-human mammalian milk. Phospholipids derived from non-human mammalian milk include phospholipids isolated from milk lipid, cream lipid, cream serum lipid, butter serum lipid, beta serum lipid, whey lipid, cheese lipid and/or buttermilk lipid. The buttermilk lipid is typically obtained during the manufacture of buttermilk. The butter serum lipid or beta serum lipid is typically obtained during the manufacture of anhydrous milk fat from cream or butter. Preferably the phospholipids are obtained from milk cream. The phospholipids are preferably derived from milk of cows, mares, sheep, goats, buffalos, horses and camels, most preferably from cow's milk. It is most preferred to use a lipid extract isolated from cow's milk. A suitable source of phospholipids derived from non-human mammalian milk is the fraction that can be isolated from milk called milk fat globule membrane (MFGM). Hence in one embodiment, the phospholipids to be used in the nutritional composition in the method or use according to the present invention are derived from or form part of the milk fat globule membrane (MFGM), or are provided as MFGM, preferably cow's milk MFGM.

The nutritional composition preferably comprises 0.5 wt % to 20 wt % phospholipid based on total lipid, preferably 0.5 wt % to 10 wt %, even more preferably 0.75 wt % to 8 wt %, even more preferably 1.2 wt % to 8 wt %, and most preferably 1.35 wt % to 5 wt % phospholipid based on total lipid.

In a preferred embodiment the lipid in the nutritional composition is in the form of lipid globules wherein:
a. the lipid globules have a mode diameter, based on volume, of at least 1.0 μm; and/or
b. at least 40 volume %, more preferably at least 45 vol %, based on total lipid volume, of the lipid globules have a diameter of 2 μm to 12 μm
   and wherein the lipid globules are at least partly coated on the surface with phospholipids.

In a more preferred embodiment, the lipid in the nutritional composition is in the form of lipid globules wherein:
a. the lipid globules have a mode diameter based on volume of at least 1.0 μm, and/or
b. at least 40 vol. %, even more preferably at least 45 vol. % of the lipid globules have a diameter of 2 to 12 μm,
   and wherein the lipid comprises at least 0.5 weight percent phospholipids based on total lipid.

By ‘coating’ is meant that the outer surface layer of the lipid globules comprises phospholipid, whereas phospholipid is much less present in the core of the lipid globule. A suitable way to determine whether phospholipid is located on the surface of lipid globules is confocal laser scanning microscopy or transmission electron microscopy; see for instance Gallier et al. (A novel infant milk formula concept: Mimicking the human milk fat globule structure, Colloids and Surfaces B: Biointerfaces 136 (2015) 329-339).

The nutritional composition preferably comprises glycerophospholipids. Examples of glycerophospholipids are phosphatidylcholine (PC), phosphatidylserine (PS), phosphatidylethanolamine (PE), phosphatidylinositol (PI) and phosphatidylglycerol (PG). Preferably the nutritional composition comprises one or more of PC, PS, PI and PE, more preferably the nutritional composition comprises at least PC.

The nutritional composition preferably comprises sphingomyelin. Sphingomyelins have a phosphorylcholine or phosphorylethanolamine molecule esterified to the 1-hydroxy group of a ceramide. They are classified as phospholipid as well as sphingolipid but are not classified as a glycerophospholipid nor as a glycosphingolipid. Preferably the nutritional composition comprises 0.05 wt % to 10 wt % sphingomyelin based on total lipid, more preferably 0.1 wt % to 5 wt %, even more preferably 0.2 wt % to 2 wt % based on total lipid. Preferably the nutritional composition comprises at least 5 wt %, more preferably 5 wt % to 40 wt % sphingomyelin based on total phospholipid, more preferably 10 wt % to 35 wt %, even more preferably 15 wt % to 35 wt %, based on total phospholipid.

The nutritional composition preferably comprises glycosphingolipids. Preferably the nutritional composition comprises 0.1 wt % to 10 wt % glycosphingolipids based on total lipid, more preferably 0.5 wt % to 5 wt %, even more preferably 2 wt % to 4 wt %, based on total lipid. The term glycosphingolipids in the present context particularly refers to glycolipids with an amino alcohol sphingosine. The sphingosine backbone is O-linked to a charged head-group such as ethanolamine, serine, or choline backbone. The backbone is also amide linked to a fatty acyl group. Glycosphingolipids are ceramides with one or more sugar residues joined in a beta-glycosidic linkage at the 1-hydroxyl position and include gangliosides. Preferably the nutritional composition contains gangliosides, more preferably at least one ganglioside selected from the group consisting of GM3 and GD3.

The nutritional composition preferably comprises phospholipid derived from mammalian milk. Preferably the nutritional composition comprises phospholipid and glycosphingolipid derived from mammalian milk. The nutritional composition preferably comprises phospholipid and optionally glycosphingolipid from mammalian milk from cows, mares, sheep, goats, buffalos, horses and/or camels. More preferably the nutritional composition comprises phospholipid and optionally glycosphingolipid from cow's milk.

Phospholipid derived from milk includes preferably phospholipid that is isolated from milk fat, cream lipid, cream serum lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipid and optionally glycosphingolipid is obtained from milk cream. Examples of suitable commercially available sources for phospholipid from milk are BAEF, SM2, SM3 and SM4 powder of Corman, Salibra of Glanbia, Lipamin M20 of Lecico, Vivinal MFGM from Friesland Campina and LacProdan MFGM-10 or PL20 of Arla.

The use of phospholipid from milk fat advantageously comprises the use of milk fat globule membranes, which are more reminiscent to the situation in human milk. The concomitant use of phospholipid derived from milk and triglycerides derived from a mix of vegetable lipid and mammalian milk fat therefore enables the manufacture of coated lipid globules with a coating more similar to human milk, while at the same time providing an optimal fatty acid profile.

Preferably the phospholipid is derived from mammalian milk fat, more preferably from cow's mammalian milk fat. Preferably the phospholipid is derived from or forms part of the milk fat globule membrane (MFGM), more preferably is derived from or forms part of cow's MFGM.

Preferably the nutritional composition comprises phospholipid and glycosphingolipid. In a preferred embodiment the weight ratio of phospholipid: glycosphingolipid is from 2:1 to 12:1, more preferably from 2:1 to 10:1 and even more preferably 2:1 to 5:1.

Methods for obtaining lipid globules with an increased size and coating with phospholipid are for example disclosed in WO 2010/027258 and WO 2010/027259.

### Digestible Carbohydrates

The powdered nutritional composition comprises digestible carbohydrates. Said digestible carbohydrates are comprised in the milk formula particles. The digestible carbohydrates preferably provide 25% to 75% of the total calories of the nutritional composition. Preferably the digestible carbohydrates provide 40% to 60% of the total calories. Based on calories the nutritional composition preferably comprises of 5 g to 20 g of digestible carbohydrates per 100 kcal, more preferably 6 g to 16 g per 100 kcal. When in liquid form, e.g., as a ready-to-feed liquid, the nutritional composition preferably comprises 3 g to 30 g digestible carbohydrate per 100 ml, more preferably 6 g to 20 g, even more preferably 7 g to 10 g per 100 ml. Based on dry weight the nutritional composition preferably comprises 20 wt % to 80 wt %, more preferably 40 wt % to 65 wt % of digestible carbohydrates. Worded alternatively, when the nutritional composition is in powder form, the digestible carbohydrates are preferably present in an amount of 20 g-80 g/100 g dry weight, more preferably 40 g-65 g/100 g dry weight.

Preferred digestible carbohydrate sources are one or more of lactose, glucose, sucrose, fructose, galactose, maltose, starch, and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt %, more preferably at least 50 wt %, more preferably at least 75 wt %, even more preferably at least 90 wt %, most preferably at least 95 wt % of the digestible carbohydrate is lactose. Based on dry weight the nutritional composition preferably comprises at least 25 wt % lactose, preferably at least 40 wt % lactose.

### Protein

The nutritional composition comprises protein. The protein preferably provides 5% to 20% of the total calories. Preferably the nutritional composition comprises protein that provides 6% to 12% of the total calories. Preferably the nutritional composition comprises less than 3.5 g protein per 100 kcal, more preferably the nutritional composition comprises between 1.5 g and 2.1 g protein per 100 kcal, even more preferably between 1.6 g and 2.0 g protein per 100 kcal. A low protein concentration advantageously is closer to human milk as human milk comprises a lower amount of protein based on total calories compared to cow's milk. The protein concentration in the nutritional composition is determined by the sum of protein, peptides, and free amino acids. Based on dry weight the nutritional composition preferably comprises less than 12 wt % protein, more preferably between 9.6 wt % and 12 wt %, even more preferably between 10 wt % and 11 wt %.

Worded alternatively, when the nutritional composition is in powder form, the proteins are preferably present in an amount of 9 g-12 g/100 g dry weight, more preferably 10 g-11 g/100 g dry weight of the composition. Based on a ready-to-drink liquid product the nutritional composition preferably comprises less than 1.5 g protein per 100 ml, more preferably between 1.2 g and 1.5 g per 100 ml, even more preferably between 1.25 g and 1.35 g per 100 ml.

The source of the protein is preferably selected in such a way that the minimum requirements for essential amino acid content are met, and satisfactory growth is ensured. Hence protein sources based on cows' milk proteins such as whey, casein, and mixtures thereof and proteins based on soy, potato or pea are preferred. In case whey proteins are used, the protein source is preferably based on acid whey or sweet whey, modified sweet whey, whey protein isolate or mixtures thereof. Preferably the nutritional composition comprises at least 3 wt % casein based on dry weight. Preferably the casein is intact and/or non-hydrolyzed.

### Non-Digestible Carbohydrates

The nutritional composition comprises in addition to the HMO particles preferably further non-digestible oligosaccharides. Preferably the powdered nutritional composition comprises non-digestible oligosaccharides with a degree of polymerization (DP) between 2 and 250, more preferably between 3 and 60. In a preferred aspect said non-digestible oligosaccharides are contained in the milk formula particles of the powdered nutritional composition.

Preferably the nutritional composition comprises as further non-digestible carbohydrates fructo-oligosaccharides, galacto-oligosaccharides and/or galacturonic acid oligosaccharides, more preferably fructo-oligosaccharides and/or galacto-oligosaccharides, even more preferably galacto-oligosaccharides, most preferably transgalacto-oligosaccharides. In a preferred embodiment the nutritional composition comprises a mixture of galacto-oligosaccharides and fructo-oligosaccharides, more preferably transgalacto-oligosaccharides and fructo-oligosaccharides.

Preferably, the nutritional composition comprises 80 mg to 2 g further non-digestible oligosaccharides per 100 ml, more preferably 150 mg to 1.5 g per 100 ml, even more preferably 300 mg to 1 g per 100 ml. Based on dry weight, the nutritional composition preferably comprises 0.25 wt % to 20 wt %, more preferably 0.5 wt % to 10 wt %, even more preferably 1.5 wt % to 7.5 wt % of non-digestible oligosaccharides. Worded alternatively, when the nutritional composition is in powder form, the non-digestible oligosaccharides are preferably present in an amount of 0.25 g-20 g/100 g dry weight, more preferably 0.5 g-10 g/100 g dry weight, even more preferably 1.5 g-8.5 g/100 g dry weight of the composition.

Preferably, the present infant formula further comprises fructo-oligosaccharides (FOS). Fructo-oligosaccharides are a non-digestible oligosaccharide (NDO) comprising a chain of beta-linked fructose units with a degree of polymerization (DP) or average DP of 2 to 250, more preferably 2 to 100, even more preferably 10 to 60. Fructo-oligosaccharide includes inulin, levan and/or a mixed type of polyfructan. An especially preferred fructo-oligosaccharide is inulin. Fructooligosaccharide suitable for use in the compositions is also commercially available, e.g., Raftiline®HP (Orafti). Preferably the fructo-oligosaccharide has an average DP above 20.

Preferably, the present infant formula further comprises galacto-oligosaccharides (GOS), preferably the galacto-oligosaccharides comprise beta-galactooligosaccharides and/or alpha galactooligosaccharides. The galacto-oligosaccharides preferably are beta-galacto-oligosaccharides. In a particularly preferred embodiment, the present infant formula comprises betagalacto-oligosaccharides ([galactose]n-glucose; wherein n is an integer ranging from 2 to 60, i.e. 2, 3, 4, 5, 6, . . . , 59,60; preferably n is selected from 2, 3, 4, 5, 6, 7, 8, 9, and 10, wherein the galactose units are in majority linked together via a beta linkage. Beta-galacto-oligosaccharides are also referred to as trans-galacto-oligosaccharides (TOS). Beta-galacto-oligosaccharides are for example sold under the trademark Vivinal™ (Borculo Domo Ingredients, Netherlands). Another suitable source is Bi2Munno (Classado). Preferably the galacto-oligosaccharides comprise beta1,3, beta-1,4 and/or beta-1,6 linkages. In a preferred embodiment, galacto-oligosaccharides comprise at least 80% beta-1,4 and beta-1,6 linkages based on total linkages, more preferably at 10 least 90%. In another preferred embodiment, the galacto-oligosaccharides comprise at least 50% beta-1,3 linkages based on total linkages, more preferably at least 60% based on total linkages. Galacto-oligosaccharides, preferably beta-galacto-oligosaccharides, are more capable of stimulating bifidobacteria. Preferably the present infant formula comprises galactooligosaccharides, preferably beta-galacto-oligosaccharides, with a degree of polymerization (DP) 15 of 2 to 10, preferably with an average DP in the range of 3 to 7. In a further embodiment, the present infant formula comprises fructo-oligosaccharides and galacto-oligosaccharides (GOS), preferably the galacto-oligosaccharides comprise beta-galactooligosaccharides. More preferably the fructo-oligosaccharides are long chain fructooligosaccharides (lcFOS) with an average DP above 20. More preferably the galacto oligosaccharides are short chain galacto-oligosaccharides (scGOS) with an average DP in the range of 3 to 7. The weight ratio of short chain galacto-oligosaccharides and long chain fructooligosaccharides ranges from 100:1 to 1:10, preferably from 20:1 to 1:1, preferably is between 7:1 and 10:1, especially about 9:1.

As the HMOS are short chain oligosaccharides, the ratio of total indigestible short chain oligosaccharide to total long chain oligosaccharides is in embodiments between 100:1 and 1:10, preferably from 20:1 to 1:1, most preferably between 8:1 and 12:1, especially about 10:1. Like for all ingredients in a nutritional composition for infants, it is possible to adjust the exact amount of HMOs dependent on the age of the infants that are fed the composition. In such an embodiment, for IMF the range is in particular about 0.5-2.0 g/l of HMOs, for follow-on formulae the range is in particular about 0.2-1.0 g/l, for young child formulae the range is in particular about 0.1-0.5 g/l of HMOs. As the amount of 2′FL is preferably between 40 and 100% based on the total HMOs, the amount of 2′FL in an IMF is preferably about 0.2-2.0 g/l, in an FOF preferably about 0.1-1.0 g/l and in a YCF preferably about 0.05-0.5 g/l. In embodiments without staging, the amount of HMOs is particularly the above-captioned levels for IMF for all ages (so these values of HMOs are also applicable in the FOF and YCF).

### Further Ingredients

In embodiments, further ingredients can be added to the base powder such as ingredients that are compatible with the lipid fraction, like in preferred embodiments, vitamins that are oil soluble. Additionally, in embodiments, vitamins, minerals and/or nucleotides can be added via dry blending. Vitamins can be selected from the group of vitamin C, any of the B vitamins, vitamin K1 and K2. The minerals can be selected from the group of: Fe, Zn, Mn, Cu, Se, I, Ca, Mg, Na, K, and P and food approved compounds containing these elements.

### Application of the Nutritional Composition

The powdered nutritional compositions of the present invention and their reconstituted ready to drink liquids are suitable for achieving beneficial effects in subjects, preferably human subjects, preferably, infants, toddlers, and children.

Accordingly, the present invention also provides powdered nutritional compositions comprising (i) milk formula particles comprising lipid, protein, digestible carbohydrates, and (ii) HMO particles, wherein the nutritional composition is selected from an infant, follow-on formula and growing up milk, wherein said nutritional composition is not human milk and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm,
   and wherein the HMO particles comprise at least 2′FL, for use in the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effect, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

Accordingly, the invention also covers the powdered nutritional compositions of the present invention and their reconstituted ready to drink liquids of the present invention for use in beneficially providing the health effects associated with both the provision of HMOs and the health effects associated with the provision of a nutritional composition comprising large lipid globules.

For some jurisdictions, the invention may also be worded as a method for (therapeutically) promoting metabolic health, promoting of the development of good body composition, preventing the development of obesity later in life, promoting balanced growth, promoting cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

For some jurisdictions, the invention may also be worded as the use of (i) milk formula particles comprising lipid, protein, digestible carbohydrates, and (ii) HMO particles, wherein the HMOs comprises at least 2′FL, in the manufacture of a powdered nutritional composition which is an infant or follow-on formula or a growing up milk, wherein the lipid in the milk formula particles comprises lipid globules and wherein
a. the lipid globules have a volume-weighted mode diameter of at least 1.0 μm, and/or
b. at least 40 vol. % of the lipid globules having a diameter of 2 to 12 μm,
   for the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

Worded differently, the invention also pertains to the use of the powdered nutritional composition according to the invention for the promotion of metabolic health, the promotion of the development of good body composition, prevention of the development of obesity later in life, the promotion of balanced growth, the promotion of lean growth, the promotion of cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving a recovery of a gut health problem.

To support that the nutritional composition are effective for use as described above, reference is made to the following documents:
1. Berger, B., et al., Linking Human Milk Oligosaccharides, Infant Fecal Community Types, and Later Risk To Require Antibiotics. mBio, 2020, 11(2).
2. Berger, P. K., et al., Human milk oligosaccharide 2′-fucosyllactose links feedings at 1 month to cognitive development at 24 months in infants of normal and overweight mothers. PLOS One, 2020, 15(2): p. e0228323.
3. Bode, L., Human milk oligosaccharides: every baby needs a sugar mama. Glycobiology, 2012, 22(9): p. 1147-62.
4. Donovan, S. M. and S. S. Comstock, Human Milk Oligosaccharides Influence Neonatal Mucosal and Systemic Immunity. Ann Nutr Metab, 2016, 69 Suppl 2: p. 42-51.
5. Natividad, J. M., et al., Blends of Human Milk Oligosaccharides Confer Intestinal Epithelial Barrier Protection in Vitro. Nutrients, 2020, 12(10).
6. Newburg, D. S., et al., Human Milk Oligosaccharides Synthetic Galactosyloligosaccharides Contain 3′-, 4-, and 6′-Galactosyllactose and Attenuate Inflammation in Human T84, NCM-460, and H4 Cells and Intestinal Tissue Ex Vivo. J Nutr, 2016, 146(2): p. 358-67.
7. Goehring, K. C., et al., Similar to Those Who Are Breastfed, Infants Fed a Formula Containing 2′-Fucosyllactose Have Lower Inflammatory Cytokines in a Randomized Controlled Trial. J Nutr, 2016, 146(12): p. 2559-2566.
8. Marriage, B. J., et al., Infants Fed a Lower Calorie Formula With 2′FL Show Growth and 2′FL Uptake Like Breast-Fed Infants. J Pediatr Gastroenterol Nutr, 2015, 61(6): p. 649-58.
9. Puccio, G., et al., Effects of Infant Formula With Human Milk Oligosaccharides on Growth and Morbidity: A Randomized Multicenter Trial. J Pediatr Gastroenterol Nutr, 2017, 64(4): p. 624-631.
10. Reverri, E. J., et al., Review of the Clinical Experiences of Feeding Infants Formula Containing the Human Milk Oligosaccharide 2′-Fucosyllactose. Nutrients, 2018, 10(10).
11. Marcobal, A., et al., Consumption of human milk oligosaccharides by gut-related microbes. J Agric Food Chem, 2010, 58(9): p. 5334-40.
12. Asakuma, S., et al., Physiology of consumption of human milk oligosaccharides by infant gut-associated bifidobacteria. J Biol Chem, 2011, 286(40): p. 34583-92.
13. Yu, Z. T., N. N. Nanthakumar, and D. S. Newburg, The Human Milk Oligosaccharide 2′-Fucosyllactose Quenches Campylobacter jejuni-Induced Inflammation in Human Epithelial Cells HEp-2 and HT-29 and in Mouse Intestinal Mucosa. J Nutr, 2016, 146(10): p. 1980-1990.
14. Newburg, D. S., et al., Innate protection conferred by fucosylated oligosaccharides of human milk against diarrhea in breastfed infants. Glycobiology, 2004, 14(3): p. 253-63.
15. Ruiz-Palacios, G. M., et al., Campylobacter jejuni binds intestinal H(O) antigen (Fuc alpha 1, 2Gal beta 1, 4GlcNAc), and fucosyloligosaccharides of human milk inhibit its binding and infection. J Biol Chem, 2003, 278(16): p. 14112-20.
16. Bienenstock, J., et al., Fucosylated but not sialylated milk oligosaccharides diminish colon motor contractions. PLOS One, 2013, 8(10): p. e76236.
17. Mezoff, E. A., et al., The human milk oligosaccharide 2′-fucosyllactose augments the adaptive response to extensive intestinal. Am J Physiol Gastrointest Liver Physiol, 2016, 310(6): p. G427-38.
18. Azagra-Boronat, I., et al., Oligosaccharides Modulate Rotavirus-Associated Dysbiosis and TLR Gene Expression in Neonatal Rats. Cells, 2019, 8(8).
19. Azagra-Boronat, I., et al., Immunomodulatory and Prebiotic Effects of 2′-Fucosyllactose in Suckling Rats. Front Immunol, 2019, 10: p. 1773.
20. Xiao, L., et al., The Combination of 2′-Fucosyllactose with Short-Chain Galacto-Oligosaccharides and Long-Chain Fructo-Oligosaccharides that Enhance Influenza Vaccine Responses Is Associated with Mucosal Immune Regulation in Mice. J Nutr, 2019, 149(5): p. 856-869.
21. Gallier S. et al., Natural and processed milk and oil body emulsions: Bioavailability, bioaccessibility and functionality. Food Structure 13, 2017, 13-23.
22. Breij L. et al., An infant formula with large, milk phospholipid-coated lipid droplets containing a mixture of dairy and vegetable lipids supports adequate growth and is well tolerated in healthy, term infants. Am J Clin Nutr, 2019; 109(3): 586-596; and the following pre-published patent applications:
WO 2010027258,
WO 2010027259,
WO 2011115490,
WO 2011115491,
WO 2012173485,
WO 2012173486,
WO 2013191542,
WO 2015065193,
WO 2016163881,
WO 2017064304, and
WO 2018104512.

### Process

The nutritional composition according to the invention can be prepared by a process in which base powder containing the milk particles is dry blended with the HMOS particles

The invention covers the nutritional composition obtainable by dry blending HMO particles, and optionally at least part of the digestible carbohydrates and optionally the indigestible carbohydrates, with a base powder that comprises the milk formula particles comprising the large lipid globules and at least part of the protein and optionally at least part of the digestible carbohydrate

In an embodiment the process to prepare the nutritional composition comprises the steps of
a) preparing a base powder wherein the digestible carbohydrates are optionally included in the base powder, added in the dry blending step as a solid or both and
b) dry blending said base powder that comprises milk formula particles comprising at least the lipid globules and protein with the HMOs particles comprising at least 2′FL.

The further ingredients may be selected from the group of vitamins, minerals, digestible carbohydrates, and further (indigestible) oligosaccharides are dry blended with the base powder and the HMOs.

The invention also covers the product obtainable by the above process. The invention also covers the product reconstituted with water or other food grade aqueous liquids that is in the form of a ready to drink liquid.

In a preferred embodiment, in the nutritional composition the amount of lipid is 10 wt %-30 wt % on dry matter, the amount of carbohydrates is 40 wt %-70 wt % on dry matter and the amount of phospholipids is 0.5 wt %-3 wt % based on total fat weight. Even more preferred in the nutritional composition the amount of lipid is 10 wt %-30 wt % on dry matter, the amount of protein is 10-30 wt % on dry matter, the amount of carbohydrates is 40 wt %-70 wt % on dry matter and the amount of phospholipids is 0.5 wt %-3 wt % based on total fat weight The process according to the invention comprises in an embodiment a process to prepare a base powder comprising at least the fat droplets, the protein and the carbohydrate and a subsequent step in which the HMOs and optionally further dry ingredients, are added by dry blending it with the base powder. In an embodiment the base powder is made by a) providing an aqueous phase with a dry matter content of 10 wt % to 60 wt % (based on total weight of the aqueous phase), which comprises at least one protein component, and optionally carbohydrate, b) providing a liquid lipid phase, which comprises at least one lipid and c) mixing the lipid phase with the aqueous phase in a ratio of 5 wt % to 50 wt % using a mixer to provide an oil water emulsion and d) drying the emulsion obtained in step c).

WO 2010027259, WO 2013135739, WO 2013135738, and WO 2016146496 disclose examples of the above process to prepare a base powder that in the process of the present invention can be used to obtain the base powder and dry blended with HMO particles.

Said process essentially comprises the following steps: a) providing an aqueous mixture comprising lipids, wherein the lipids comprise 50 to 100 wt % vegetable lipid based on total lipid and wherein 0.2 to 20 wt % based on total lipid is phospholipid, and comprising protein, digestible carbohydrate, and optionally non-digestible oligosaccharides, and b) homogenizing said mixture in two steps with preferably 5-100 bar, more preferably 30-100 bar in a first step and 5-50 bar in a second step, and c) preferably sterilizing said homogenizing mixture and d) preferably spray drying said sterilized mixture. In the context of the present invention these steps are preferably followed by dry blending said base powder or milk formula particles with HMO particles.

Alternatively, the process may be described as a two-step emulsification process by the following steps:
a) providing an aqueous phase with a dry matter content of 5 to 75 wt. % (based on total weight of the aqueous phase), which comprises at least one protein component, b) providing a liquid lipid phase, which comprises at least one lipid and c) carrying out a first homogenization step by homogenizing the lipid phase with the aqueous phase in a ratio of 3 to 50% (w/w) so as to obtain a first lipid and protein component-containing composition comprising lipid globules, wherein at least 10 vol.-% of the lipid globules have a diameter of >12 μm and/or wherein the lipid globules have a volume-weighted mode diameter from 5 to 25 μm, d) carrying out a second homogenization step by homogenizing the first lipid and protein component-containing composition obtained in step c) with an atomizer, wherein the particle size of the lipid globules obtained in step c) is reduced so as to obtain a second lipid and protein component-containing composition comprising lipid globules, wherein less than 10 vol.-% of the lipid globules have a diameter of >12 μm and/or wherein the lipid globules have a volume-weighted mode diameter from 2.5 to 7 μm. In the context of the present invention these steps are preferably followed by dry blending said base powder with HMOs.

### Use

A further aspect of the invention relates to the use of milk formula particles to reduce powder segregation in a powdered nutritional composition comprising (i) milk formula particles and (ii) HMO particles, wherein the milk formula particles comprise digestible carbohydrates, lipid, and protein, and wherein the lipid in the milk formula particles is in the form of lipid globules, and
a. the lipid globules have a mode diameter based on volume of at least 1 μm, and/or
b. at least 40 vol. % of the lipid globules have a diameter of 2 to 12 μm, wherein the HMO particles comprise at least 2′fucosyllactose (2′FL).

Preferably, the milk formula particles and the HMO particles differ in size, more preferably they differ in their Dx(10), Dx(50) and/or Dx(90).

### EXAMPLES

### Example 1

### Materials

A small blend is made using base powder (BP) according to the invention prepared in a similar way as in Example 1B of WO 2010/027259 wherein the fat globules have a volume weighted mode diameter that lies between 3 and 7 μm, that is dry blended with lactose (FrieslandCampina Veghel), premix (a mixture of powdered vitamins and minerals) and 2′FL (GlyCare 2FL 9000, DSM). As a reference a standard infant milk base powder (BP with substantially the same composition having fat globules with a volume weighted mode diameter that lies between 0.3 μm and 0.5 μm) is also mixed with the same ingredients and tested for segregation. The amount of material used for the blends is shown in Table 1.

TABLE 1
Amount of ingredients used for 2′FL

| | Reference | Invention |
|---|---|---|
| Ingredient | (g) | (g) |
| Comparative Infant Milk Base powder | 82.005 | |
| with small fat globules | | |
| Base powder with large fat globules | | 82.055 |
| Lactose (FrieslandCampina Veghel) | 15.338 | 15.352 |
| Premix (mix of vitamin and minerals) | 1.796 | 1.797 |
| 2′FL (DSM GlyCom) | 0.888 | 0.850 |

### Method

For the physical analysis, the segregation risk of the different blends was assessed. The Jenike & Johanson segregation tester was used to assess segregation using the method described in ASTM D6941-12. The goal of the fluidization segregation test is to bring the material in the test chamber to a completely fluidized/aerated state, then allow slow deaeration (settlement) of the powder. When the test is finished and all powder in the chamber settled, samples can be collected. The column of powder is split in three sections and each section (top, middle, bottom) can be analysed for segregation.

Both the top and the bottom sample as well as the original sample (prior to being fluidized) were characterized in terms of particle size distribution (PSD) by means of the Malvern Mastersizer 3000.

From the PSD, three characteristic parameters: d10, d50 and d90 were recorded for each of the samples. The segregation potential (SP) is then calculated as:

$$S P = \frac{{d}_{top} - {d}_{b o t t o m}}{{d}_{0}}$$

Where d is any of the three quantiles (d₁₀, d₅₀ or d₉₀, all by volume). A lower value for SP indicated less segregation potential. The sub-index bottom and top refer to the separation done after fluidization, while d₀ refers to the quantile value prior to the powder being admitted to the test chamber.

The results of the test are shown in Table 2. From the table it can be seen that segregation does not occur with the product of the invention, indicated by the SP values of SP_{d10}, SP_{d50}, SP_{d90} of 1%, 1% and 6% respectively.

TABLE 2
Segregation potential results

| Sample Name | SP_{d10} | SP_{d50} | SP_{d90} |
|---|---|---|---|
| Reference | 11% | 21% | 19% |
| Invention | 1% | 1% | 6% |

### Example 2

The particle size distribution of 2′-FL particles (DSM), a reference infant milk base powder with small lipid globules (reference) and the powder according to the invention with large lipid globules (Invention) was determined (table 3).

Particle size distribution was measured using laser diffraction (Malvern Mastersizer 3000) equipped with a dry dispersion unit (Aero S) operated at 1 bar dispersion pressure. Optical properties for evaluation of data were 1,52/0,1 and using non-spherical particle type.

Using the particle size distribution values, in addition the Euclidian difference between the powder according to the invention and the 2′-FL particles as well as between the reference powder and the 2′-FL particles was determined (table 4) according to the following mathematical formula:

$$ND ( a , b ) = \frac{1}{D - 1} \sqrt{{\sum }_{i = 1}^{D} {( {a}_{i} - {b}_{i} )}^{2}} \qquad ( I )$$

The Euclidian difference indicates how similar two distributions are by using said mathematical expression (Euclidean distance). The greater the value is, the more different the distributions are.

Particle size distribution of the powder according to the invention was significantly larger; the spread in Dx(10) to Dx(90) is 355 μm for the powder according to the invention as compared to the reference powder product having a spread of 153 μm. The Dx(50) of the powder according to the invention was also more than double the size of the Dx(50) of the reference powder.

The Euclidean difference of the milk formula powder according to the invention and 2′-FL was found significantly higher that the Reference powder and 2′-FL. The higher Euclidean difference of the milk formula powder according to the invention and 2′-FL shows that the 2 powders are very different in terms of size.

Notwithstanding the difference in the milk formula particles according to the invention and the HMO particles the latter combination beneficially did not segregate.

TABLE 3
Quantiles of powder particle size distribution

| | | Invention | | Reference | | 2′FL |
|---|---|---|---|---|---|---|
| Dx | (10) | 72 | μm | 41 | μm | 20 μm |
| Dx | (20) | 130 | μm | 57 | μm | 32 μm |
| Dx | (50) | 230 | μm | 98 | μm | 53 μm |
| Dx | (80) | 351 | μm | 157 | μm | 78 μm |
| Dx | (90) | 427 | μm | 194 | μm | 93 μm |

TABLE 4
Euclidian difference of the powders in combination with 2′-FL

| | Euclidean |
|---|---|
| Combination | difference |
| Invention + 2-FL | 0.329 |
| Reference + 2-FL | 0.200 |

1. Powdered nutritional composition comprising (i) milk formula particles comprising lipid, protein, digestible carbohydrates, and (ii) human milk oligosaccharides (HMO) particles,
wherein the nutritional composition is selected from an infant, follow-on formula and growing up milk, wherein said nutritional composition is not human milk and
wherein the lipid in the milk formula particles is in the form of lipid globules containing a coating comprising phospholipids and
a. the lipid globules have a mode diameter based on volume of at least 1 μm, and/or
b. at least 40 vol. % of the lipid globules have a diameter of 2 to 12 μm, and
wherein the HMOs particles comprise at least 2′fucosyllactose (2′FL), and
wherein the (i) milk formula particles have a powder particle size distribution comprising a Dx(50) of 200 μm to 300 μm, and
wherein the (ii) HMO particles have a powder particle size distribution comprising a Dx(50) of 40 μm to 130 μm, and
wherein the phospholipids are derived from cow's milk.

2. The powdered nutritional composition according to claim 1, wherein the milk formula particles have a powder particle size distribution comprising a Dx(10) of at least 60 μm and/or a Dx(90) of at least 400 μm.

3. The powdered nutritional composition according to claim 1, wherein HMO particles have a powder particle size distribution comprising a Dx(10) of at least 5 μm and/or a Dx(90) of at most 315 μm.

4. The powdered nutritional composition according to claim 1, wherein the (i) milk formula particles and the (ii) HMO particles have an Euclidean difference of more than 0.2.

5. The powdered nutritional composition according to claim 1, wherein the difference between the Dx(20) of the milk formula particles and the Dx(80) of the HMO particles is at least 10 μm.

6. The powdered nutritional composition according to claim 1, wherein the amount of phospholipids is 0.5-20 wt % on total lipid content.

7. The powdered nutritional according to claim 6, wherein the phospholipids are derived from cow's milk fat globule membrane (MFGM) or are provided as cow's milk MFGM.

8. The powdered nutritional composition according to claim 1, wherein i) the lipid comprises linoleic acid and alpha-linolenic acid in a weight ratio of 2 to 20, and/or ii) the lipid comprises at least 10 weight percent palmitic acid based on total lipid, and at least 15 weight percent of this palmitic acid is esterified to the sn-2 position of a triglyceride based on total palmitic acid.

9. The powdered nutritional composition according to claim 1, wherein the (ii) HMO particles are present in an amount of 0.1 wt %-2 wt % based on dry matter weight.

10. The powdered nutritional composition according to claim 1, wherein the lipid contains both vegetable fat and milk fat.

11. The powdered nutritional composition according to claim 1, wherein the amount of lipid is 10 wt %-30 wt % on dry matter, the amount of protein is 9.6 wt %-12 wt % on dry matter, the amount of carbohydrates is 40 wt %-70 wt % on dry matter and the amount of phospholipids is 0.5 wt %-3 wt % based on total fat weight.

12. The powdered nutritional composition according to claim 1, wherein the HMOs comprise 2′FL and at least one selected from lacto-N-neotetraose (LNnT), para-lacto-N-neohexaose (para-LNnH), sialic acid, 3′ sialyllactose (3′SL) and 6′ sialyllactose (6′SL), 3′fucosyllactose (3-FL), difucosyllactose (DFL), lacto-N-fucopentaose (LNFP such as lacto-N-fucopentaose I, lacto-N-fucopentaose II, lacto-N-fucopentaose III, lacto-NfucopentaoseV), lacto-N-fucohexaose, lacto-N-difucohexaose (LNDFH such as lacto-N-difucohexaose I and lacto-N-difucohexaose II), sialyl-lacto-N-tetraose (LSTa), sialyl-lacto-N-tetraose b (LSTb), sialyl-lacto-N-tetraose c (LSTc) disialyllacto-N-tetraose (DSLNT) Lacto-N-neodifucohexaose (LNnDFH I), fucosyllacto-Nhexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose and tri-fuco-para-Lacto-N-hexaose I, lacto-N-tetraose (LNT), or any combination thereof.

13. The powdered nutritional composition according to claim 1, in addition containing further indigestible carbohydrates, preferably one or more from the group of FOS and GOS carbohydrates.

14. The powdered nutritional composition according to claim 1, obtainable by dry blending HMO particles, and optionally at least part of the digestible carbohydrates and optionally the indigestible carbohydrates, with a base powder that comprises the large lipid globules and at least part of the protein and optionally at least part of the digestible carbohydrates.

15. The powdered nutritional composition according to claim 1, wherein the base powder is obtainable by dosing the lipid to an aqueous phase comprising protein and optionally part of the digestible carbohydrates and at least part of the phospholipids, mixing the combined aqueous and lipid phase to obtain an emulsion, and subsequently drying the emulsion, preferably in a spray dryer.

16. A process to prepare the nutritional composition of claim 1, comprising the steps of
a) preparing a base powder wherein the digestible carbohydrates are optionally included in the base powder, added in the dry blending step as a solid or both and
b) dry blending said base powder comprising at least the lipid globules and protein with the HMO particles comprising at least 2′FL.

17. The process according to claim 16, wherein the base powder is made by a) providing an aqueous phase with a dry matter content of 10 wt % to 60 wt % (based on total weight of the aqueous phase), which comprises at least one protein component, b) providing a liquid lipid phase, which comprises at least one lipid and c) mixing the lipid phase with the aqueous phase in a ratio of 5 wt % to 50 wt % using a mixer to provide an oil water emulsion and d) drying the emulsion obtained in step c).

18. A process according to claim 16, wherein further ingredients selected from the group of vitamins, minerals, digestible carbohydrates, and further oligosaccharides, preferably indigestible oligosaccharides, are dry blended with the base powder and the HMOs.

19. The powdered nutritional composition according to claim 1, wherein the powdered nutritional composition is reconstituted with water or other food grade aqueous liquid, to form a ready-to drink liquid.

20. A method for promoting metabolic health, promoting the development of good body composition, preventing of the development of obesity later in life, promoting balanced growth, promoting lean growth, promoting cognitive development, improving brain health, improving gut health, providing beneficial prebiotic effects, increasing immune cell function and immune health, preventing infections or improving the recovery from infections, stimulating intestinal barrier functions/epithelial cell modulators and so to improve gut health and reducing the risk of gut health problems and/or improving recovery of a gut health problem, the method comprising administering the nutritional composition of claim 1.
